(19) United States
(12) Patent Application Publication
(10) Pub. No.: US 2024/0321395 A1
(43) Pub. Date: Sep. 26, 2024

(54) MITOCHONDRIAL PROBES FOR ENDOGENOUS CONTROL AND CONTAMINATION DETECTION

(71) Applicant: Helix, Inc., San Mateo, CA (US)

(72) Inventors: Dana Wyman, San Diego, CA (US); Tracy Basler, San Diego, CA (US); Andrew Dei Rossi, San Carlos, CA (US); Shishi Luo, San Francisco, CA (US); William Lee, Mountain View, CA (US); Efren Sandoval, Poway, CA (US)

(21) Appl. No.: 18/124,670

(22) Filed: Mar. 22, 2023

(51) Int. Cl. G16B 30/00; C12Q 1/6869; G16B 20/00

(52) U.S. Cl. CPC G16B 30/00; C12Q 1/6869; G16B 20/00

(57) ABSTRACT

Various embodiments disclosed relate to a method of quality control for a biological sample. The method can include receiving genetic data sequenced from the biological sample, determining whether an endogenous control is apparent in the genetic data, and in the event that the endogenous control is apparent in the genetic data, determining whether the biological sample is contaminated. The genetic data can be analyzed for sequences from both pathogen genetic material and mitochondrial genetic material. The endogenous control can be determined based on whether at least a threshold amount of the genetic data includes sequences for the mitochondrial genetic material.

### BACKGROUND

Biological samples can be analyzed for pathogens, such as viruses, through the use of assay panels. Such assay panels can be designed to collect genetic material from a larger number of pathogens simultaneously. For example, a respiratory viral assay may include probes directed to upwards of twenty-five or thirty-five specific viruses of interest. Once the targeted genetic material is collected through the assay probes, such genetic material can be sequenced. The sequencing data can then be analyzed in bioinformatic processes to provide valuable biological information. When such an assay is used, a control can be used during the sequencing and analysis to confirm that the biological sample is indeed a sample from a human patient.

### SUMMARY OF THE DISCLOSURE

In an example, a method of performing quality control for a biological sample can include: receiving genetic data sequenced from the biological sample, wherein the genetic data has been analyzed for both pathogen genetic material and mitochondrial genetic material; determining whether an endogenous control is apparent in the genetic data based on whether at least a threshold amount of the genetic data includes sequences for the mitochondrial genetic material; and in the event that the endogenous control is apparent in the genetic data, determining whether the biological sample is contaminated by calculating a composite alternative allele fraction of the sequences for the mitochondrial genetic material.

In an example, a system for quality control of a biological sample includes a processor and a memory that holds instructions which when executed cause the processor to: access genetic data sequenced from the biological sample, wherein the genetic data has been analyzed for both pathogen genetic material and mitochondrial genetic material; determine whether an endogenous control is apparent in the genetic data based on whether at least a threshold amount of the genetic data includes sequences for the mitochondrial genetic material; calculate a composite alternative allele fraction of the sequences for the mitochondrial genetic material; and determine whether the biological sample is contaminated based on the calculated composite alternative allele fraction.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A depicts a diagram of an example bioinformatics analysis system.

FIG. 1B is a flow chart illustrating a method of using a bioinformatics analysis system.

FIG. 2A is a flow chart illustrating a method of intaking and preparing a genetic sample in a wet laboratory for bioinformatic analysis in an example.

FIG. 2B is a flow chart illustrating a method of intaking a genetic sample in a wet laboratory for bioinformatic analysis in an example.

FIG. 2C is a flow chart illustrating a method of preparing a genetic sample in a wet laboratory for bioinformatic analysis in an example.

FIG. 3A is a flow chart illustrating a method of bioinformatic analysis of a genetic sample in an example.

FIG. 3B is a flow chart illustrating a method of bioinformatic analysis of a genetic sample in an example.

FIG. 3C is a flow chart illustrating a method of bioinformatic analysis of a genetic sample in an example.

FIG. 3D is a flow chart illustrating a method of bioinformatic analysis of a genetic sample in an example.

FIG. 3E is a flow chart illustrating a method of bioinformatic analysis of a genetic sample in an example.

FIG. 4 depicts a computer system in an example.

FIG. 5 is a graph illustrating alternative allele fractions in an example.

FIG. 6A is a graph illustrating median alternative allele fractions in an example.

FIG. 6B is a graph illustrating median alternative allele fractions in an example.

### DETAILED DESCRIPTION

Discussed herein is a method of using mitochondrial DNA (mtDNA) to produce an endogenous control for a genetic sample, and a method of using mtDNA for distinguishing contaminated samples. The method includes a wet lab chemical approach using mitochondrial probes to collect mtDNA alongside collection of pathogen genetic material, sequencing that genetic material including both pathogen genetic material and mtDNA, and analyzing the sequence data in a bioinformatic analysis to help determine whether the biological sample is indeed a human sample, and whether the biological sample is likely contaminated.

Human mtDNA is inherited maternally, and is haploid in nature. The presence of mtDNA in a biological sample can be indicative that the biological sample was, indeed, taken from a human patient. In this way, mtDNA can act as an endogenous control. Moreover, when mtDNA is sequenced, the genetic material should not contain heterozygosity (i.e., owing to its haploid nature). Accordingly, when mtDNA is analyzed, the alternative allele fractions of any variants therein should be close to 1, with the rare exception of noise, sequencing error, or other artifacts. If a sequenced mtDNA sample contains a large number of variants, such as indicated by an alternative allele fraction analysis, the sample likely contains mtDNA from more than one individual patient. In this case, the sample may be contaminated. Thus, mtDNA can be used both as an endogenous control, and for detection of contamination in a biological sample.

Discussed herein, a method can include using mitochondrial probes during a viral panel assay, such as to simultaneously collect and analyze mtDNA along with pathogen genetic material of interest. The methods discussed herein use mitochondrial probes to provide an endogenous control and allow for distinguishing between contamination or coinfection in a sample, leveraging the sequencing and analysis of mtDNA captured. This method provides a number of advantages.

First, the approach allows for naturally occurring mtDNA, already present in the biological samples being analyzed for pathogen genetic material, to act as both an endogenous control and a means of contamination detection. This can be done contemporaneously with a pathogen panel assay, such as a viral respiratory panel.

Second, the use of mtDNA as an endogenous control can allow for distinguishing a true viral negative sample from an assay failure (or empty well) without running independent orthogonal testing, such as a separate round of quantitative polymerase chain reaction (qPCR) for each pathogen being considered in the panel. This helps save time and reduce the cumbersome nature of running multiple qPCR for each sample to provide a control.

Third, the mtDNA based approach allows for detection of contamination between any two samples with human material, regardless of whether the samples are for the same pathogen, a different pathogen, or even if no pathogen is present. This helps detect contamination at a much higher level than pathogen centered alternative allele fraction based techniques.

Fourth, by using mitochondrial coverage for both the endogenous control and contamination detection, fewer regions of the human genome need to be targeted overall, reducing the complexity of the assay.

Endogenous Control. A variety of viral sequencing assays are used when analyzing genetic samples and in preparation for bioinformatic analysis. Such viral assays require a control, such as an endogenous control, to confirm that the biological sample being sequenced did indeed come from a human patient. This helps the researcher validate that the sample is useable for bioinformatic analysis.

In some cases, viral sequencing assays are limited, and directed to a single, specific pathogen or genetic area of interest. For example, viral sequencing of COVID-19 (e.g., the SARS-CoV-2 virus), can be done with a viral sequencing assay directed to that virus. In this case, to assess a control and confirm whether the sample can continue to be used for bioinformatic analysis, qPCR can be run on the sample to produce a quantification cycle (Cq) value. The Cq value can be used to determine whether the sample “passes” or “fails”. If the sample passes, a control is satisfied, and it can be used in further bioinformatic analysis. If the sample fails, the sample is not useable for further bioinformatic analysis. A variety of circumstances can cause the control, indicated by the Cq value, to fail. In an example, the sample may have low viral titer, resulting in a negative Cq value. Such a sample would fail. In some cases, a sample with a high viral titer can fail according to its Cq value. In this case, there may be a problem in the assay itself. In either case, the Cq value, correlated to the specific viral assay, can be used to help determine whether a control is satisfied and the sample is useable for bioinformatic analysis. In this way, the Cq value can be used as a control for a single-pathogen or limited assay. While this Cq and qPCR based technique can be applied to simple assays for analyzing a single pathogen of interest, it is more challenging to apply such a qPCR and Cq analysis to a larger assay panel directed to multiple pathogens.

For example, where a sample is being analyzed with a full respiratory panel sequencing assay, a large number of viruses are often assessed at the same time, instead of a single virus (e.g., the SARS-COV-2 virus discussed above). In this case, to obtain Cq values for each virus in the panel would be cumbersome, and would involve designing, validating, and performing qPCR for each pathogen in the assay every time a sample needed to be validated and a control needed to be produced. When a single respiratory panel contains many viral targets (for example, 30+ targets) this quickly becomes unsustainable. In particular, if a full respiratory panel is applied to a sample where it is unknown if or what specific viral infections are present, a number of negative Cq values may occur if this type of control method is used. By comparison, as discussed herein, an endogenous control could be used to distinguish between true negatives and possible assay failures.

Moreover, a panel assay, directed to multiple pathogens (such as a viral respiratory assay) requires a control to show the biological sample being tested is, indeed, a biological sample from a human patient. In such cases, a spiked positive control (e.g., containing an artificial amount of analyte) could be used, but that would not prove that a biological sample from a human patient is being tested. To show an actual biological sample from a human patient is being tested, probes can be used that target human genetic material (as opposed to probes that target pathogen genetic material). The human genetic material would thus be sequenced alongside any targeted pathogen genetic material in the sample, even if the sample is negative for pathogen genetic material.

In the methods discussed herein, mitochondrial probes can be used to produce an endogenous control for such a biological sample. The mitochondrial probes can capture human genetic data in the form of mtDNA. The haploid nature of mtDNA allows for particular benefits in distinguishing an endogenous control and validating the biological sample.

Sample Contamination. Additionally, contamination is a concern with reporting viral sequencing results. For example, where a larger respiratory panel assay is used, it is important to assess a situation where multiple samples may have been mixed together. Additionally, if two samples involved in such a contamination event contain the same pathogen, any consensus sequence generated from that contaminated sample may provide misleading data for bioinformatic analysis.

When a viral sequencing assay directed to a single, specific pathogen or genetic area of interest is used, such as the viral sequencing of COVID-19 discussed above, contamination can occur, and alternative allele fractions or frequencies of variants of that virus could be used to screen for possible contamination. In this case, because the viral genome is haploid, calls of alternative allele fractions should be homozygous. If the calls of the alternative allele fractions are not homozygous, the sample may be contaminated.

But this type of pathogen alternative allele fraction based method of detecting contamination is limited and not applicable to larger viral assays that involve multiple pathogens. Because this approach relies on computations at the level of a single viral genome, it is not informative when multiple pathogens are being analyzed simultaneously and does not provide information on whether contamination is present. Moreover, with regards to SARS-COV-2 specifically, multiple variants have and could in the future sweep through the population at the same time. When the SARS-COV-2 sequences present in different patients are very similar, it becomes more difficult to detect contamination events between them on the basis of pathogen alternative allele frequency. All these factors indicate that a pathogen targeted alternative allele fraction based method of detecting contamination is not applicable to a large viral assay panel, such as a full respiratory panel. Tracking contamination on a wider scale, such as when these large viral assay panels are used, would be beneficial. However, sequencing a large amount of human genetic material alongside the pathogen genetic material of interest would be time consuming and complex. This could additionally be confounded by variable expression levels of human RNA targets, leading to inconsistent performance. For this reason, the use of mtDNA is particularly appealing, as it avoids some of these pitfalls.

Thus, discussed herein, the use of mitochondrial probes to gather mitochondrial genetic material can help to provide information to help detect contamination. For example, the mitochondrial probes can be used to intentionally target certain regions of the human genome (e.g., mtDNA) and screen variant calls in that region for genetic evidence of more than one human patient. In an example, the mtDNA in the sample can be collected, sequenced, and analyzed to determine if there is mtDNA indicating more than one human patient in the sample. If so, the sample is likely contaminated.

### Definitions

As used herein, “alternative allele fraction”, or “alternate allele fraction” is the number of reads supporting an alternate allele (such as a mutation) divided by the total number of reads covering the position.

As used herein, “average alternative allele fraction,” “median alternative allele fraction”, “mean alternative allele fraction”, or “weighted alternative allele fraction” is calculated respectively as the average, median, mean, or weighted value of alternate allele fractions at sites where at least a threshold, e.g., 15%, of the reads support a mutation. These terms may be generally referred to as “composite alternative allele fractions.”

As used herein, “accession”, or “accessioning” refers to receiving and preparing a sample for later laboratory processes.

As used herein, “amplifying” refers to the production of multiple copies of a sequence of nucleic acid or other genetic material, such as RNA or DNA.

As used herein, “bioinformatics” refers to the science of collecting complex biological data such as genetic codes.

As used herein, “biological sample”, or “sample” refers to a specimen from a patient, such as for bioinformatic research.

As used herein, “calling” an allele can include identifying one or more alleles, such as alternative alleles or mutations, at a particular locus of sequenced genetic material.

As used herein, “contamination” refers to a sample that is impure, polluted, or unsuitable for biological analysis and research.

As used herein, “Cq value” is the inverse to the amount of target nucleic acid that is in the sample, and correlates to the number of target copies in a sample.

As used herein, “endogenous control” refers to sequence data whose expression level should not differ between samples, such as a housekeeping or maintenance gene.

As used herein, “genetic material” refers to a fragment, molecule, or a group of nucleic acids, such as DNA or RNA, or other genetic material, such as mitochondrial genetic material.

As used herein, “haploid” refers to genetic material having a single set of unpaired chromosomes.

As used herein, “locus” or “loci” refers to the position of a gene or mutation on a chromosome or on a fragment of genetic material.

As used herein, “mtDNA” or “mitochondrial DNA” refers to genetic material from mitochondria.

As used herein, “mutation” refers to a changed structure of a gene that results in a variant form of the gene (e.g., with respect to a reference genome).

As used herein, “pathogen” refers to a bacterium, virus, or other microorganism that can cause disease.

As used herein, “qPCR” refers to quantitative polymerase chain reaction used for measuring DNA via polymerase chain reaction amplification techniques.

As used herein, “read” or “read pair” refers to data that defines a DNA or RNA sequence from one fragment or small section of genetic material.

As used herein, “recombinant” refers to genetic material formed by recombination (e.g., recombination of genetic material from two or more different variants).

As used herein, “sequencing” refers to a process of determining the nucleic acid sequence, the order of nucleotides in genetic material.

As used herein, “variant” or “genetic variant” refers to a subtype of a microorganism that is genetically distinct from other subtypes.

As used herein, “viral titer” an assay used to assess viral level.

As used herein “walking” a sequence of genetic material can include reviewing and reading a sequenced portion of genetic material from the 5′end towards the 3′end to determine whether particular genetic markers, such as mutations, are present.

### Bioinformatic Analysis System and Methods

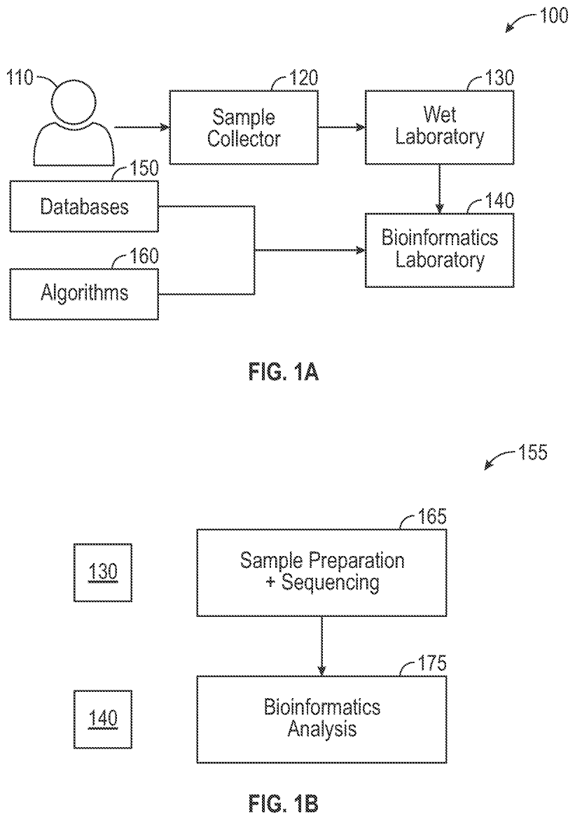

FIG. 1A depicts a diagram of an example bioinformatics analysis system 100, while FIG. 1B is a flow chart illustrating a method 155 of using such a bioinformatics analysis system 100.

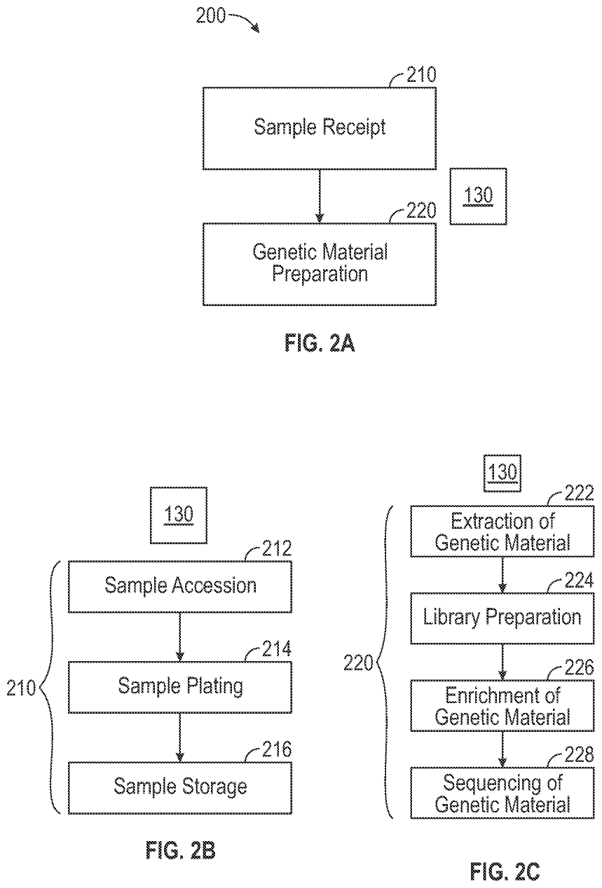

The system 100 can include both physical or “wet” laboratory components, and bioinformatics components. For example, the system 100 can interact with patients 110, from whom biological samples can be collected, in addition to sample collectors 120, which may be, for example, doctors, pharmacies, or other appropriate places where patient samples can be taken. The system 100 includes a wet laboratory 130 which is positioned to receive the biological samples and process those samples to produce sequenced genetic material for analysis, such as at step 165 of method 155. These methods of sample receipt, handling (e.g., accession), and sequencing, are discussed in detail below with reference to FIGS. 2A to 2C.

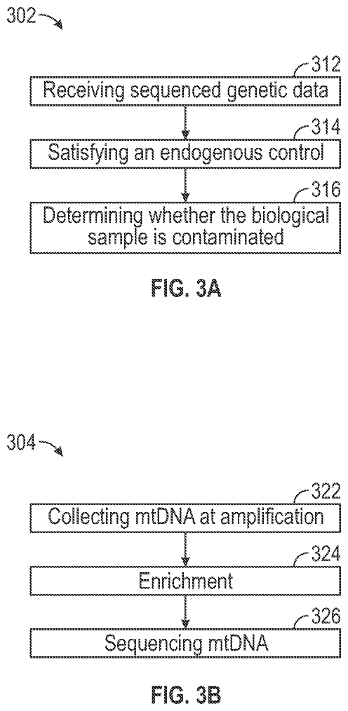

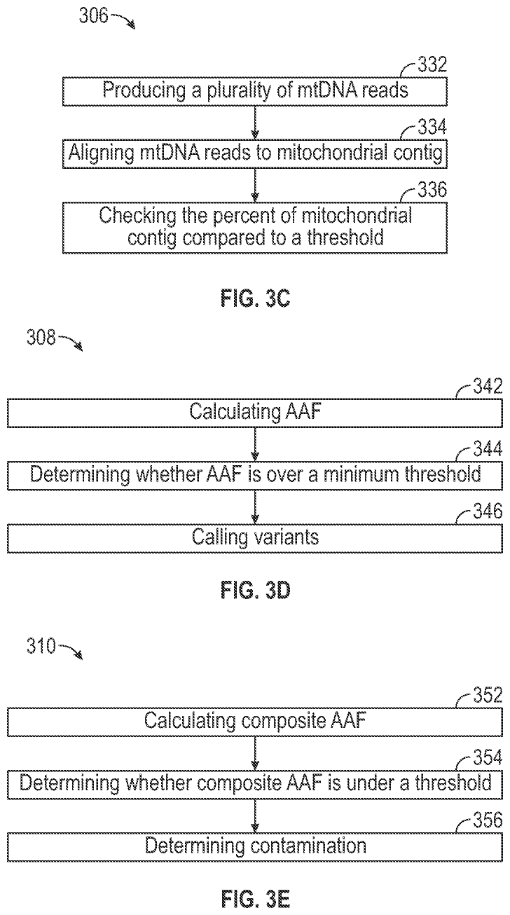

The system 100 can additionally include data driven components, such as databases 150 and algorithms 160 or other programs that support the bioinformatic laboratory 140 used to analyze genetic information. These data driven components can be used to do bioinformatic analysis (step 175 in method 155). Specific examples of such bioinformatic analysis are discussed in detail below with reference to FIGS. 3A to 3C.

### Sample Processing Methodology

Before bioinformatic analysis, biological samples are collected and sequenced through physical components of the system 100, such as through a wet laboratory 130. Methods of receiving and processing such samples are summarized in FIGS. 2A to 2C. FIG. 2A is a flow chart illustrating a method 200 of intaking and preparing a genetic sample in a wet laboratory for bioinformatic analysis. The method 200 can include two primary portions: receiving the samples (step 210) and preparing genetic material (step 220). FIG. 2B illustrates portions of step 210, including a method of intaking a genetic sample in a wet laboratory for bioinformatic analysis. FIG. 2C illustrates the portions of step 220 of the method 200, a method of preparing a genetic sample in a wet laboratory for bioinformatic analysis.

The method 200 can begin with sample collection. For example, the samples can be collected by receiving a nasal swab, blood, saliva, or other material potentially containing genetic material indicative of a pathogen. The pathogen under study can be, for example, an RNA virus such as SARS-COV-2 or HIV, an adenovirus, or another type of pathogen with multiple variants having genetic material that could recombine for recombination and coinfection analysis.

Accessioning Samples. Once received at the laboratory, at step 212, the samples can be accessioned, that is, prepared for later laboratory processes. For example, accessioning can include receiving a batch of samples. A batch of samples can include, for example, hundreds of individual samples, or thousands of individual samples. Each sample can be retained in a sample container. For example, test tubes can be used to store each of the samples. The sample containers can be sealed to help prevent environmental exposure and prevent sample co-mingling. For example, the sample containers may be sealed via a cap that is threaded, glued, press-fit, or otherwise affixed via appropriate sealing mechanism. When the samples are received in a batch, the corresponding sample containers may also include one or more remnants of a sampling tool, such as a swab used to collect the sample.

In some cases, the sample containers may be accompanied by Customer Sample Identifiers (CSI) such as by a component affixed to or integrated with the sample container. Such a CSI can uniquely distinguish individual sample containers from other sample containers being received. For example, a CSI may uniquely distinguish a sample from other samples in the same batch, other samples received on the same date, or other samples received from the same customer. Such CSI can be provided as a label such as a bar code or a Quick Response (QR) code, a chip such as a Radio Frequency Identifier (RFID), or another type of visual, transmission-generating, or other component affixed to or integrated with the sample container.

In some cases, the sample containers can be further sealed in an external container, such as a bag. External containers can help prevent contamination of samples, such as by preventing biological material from the samples contacting other or external surfaces. An external container can also help prevent cross-contamination between samples. Moreover, when a sample includes blood or a pathogen, the external container can provide an additional barrier to protect technicians who may handle the samples. The external container can additionally include documentation correlating to the CSI, such as information on the patient that the sample was sourced from, information indicating circumstances of sampling, for example, a sampling date, a sampling method, a location that the sample was acquired, a name or title for a person who performed the sampling, other information, or combinations thereof.

In some cases, the samples can be in a chemical solution. For example, the sample may be prepared in an aqueous solution, such as a saline solution. In some cases, the samples can include a bodily fluid such as saliva, mucus, blood, or other. In an example, the sample can have a volume of about 2 mL, of about 3 mL, of about 4 mL, or of about 5 mL.

The samples include genetic material. For example, the samples can include Deoxyribonucleic Acid (DNA) or Ribonucleic Acid (RNA). In an example, the genetic material is one or more of many constituent components within the sample. For example, one portion of the genetic material may exist within the nuclei or mitochondria of white blood cells that are included within the sample. In another example, another portion of the genetic material may exist within viruses or bacteria within the sample. In these types of examples, the genetic material is not yet isolated from the remaining constituent components of the sample. Thus, the genetic material should be isolated.

To begin isolating the genetic material, batches of the samples can be heated in ovens to facilitate cell lysis. The temperature and duration of heating can be chosen such that pathogenic material within the samples is rendered harmless, such that cellular lysis occurs, or both. For example, the samples can be heated at a temperature of between about 40° C. and 80° C., or at a temperature of between about 15° C. and 200° C., or at another appropriate temperature range. The samples can be heated for a time period of about 30 minutes, or for a time period of about 50 minutes, or for another appropriate time period. In some cases, such as where the samples are the contents of a blood draw, the heating step may be skipped.

After heating, the batches of samples can be removed from the ovens. In an example, sample containers can be removed from external containers, such as by cutting open the external containers. The sample containers can be inspected, either in a manual, automated, or semi-automated fashion. For example, a technician or an automated system can determine the CSI for the sample and compare the CSI to documentation accompanying the batch. If there is a discrepancy between the CSIs on the sample container and in the documentation, the sample may be flagged as having an error condition. Similarly, if the CSI on the sample container is damaged (such as by abrasion, heat-damage, or water-damage) and has become unreadable, the sample may be flagged as having an error condition.

In some cases, the technician or automated system can further inspect the contents of the sample container, such as visually. If the sample does not include expected constituent components, then the sample can be flagged as having an error condition. For example, if the sample includes a fluid that is not permitted (such as extraneous blood), includes an entire swab or no swab, is within a fractured or broken sample container, or is outside of an expected range of volume (e.g., between two and five milliliters), or other conditions, then the sample can be flagged as having an error condition.

Subsequently, samples that have not been flagged with an error condition can proceed to sample integration. Here, the sample can be assigned a Laboratory Sample Identifier (LSI). Such an LSI can uniquely identify the sample from other samples received in the same batch, received on the same day, processed in the same laboratory, handled by the same company for sequencing, or combinations thereof. The LSI can be stored in a laboratory sample database, and uniquely correlated to the CSI for the sample. The LSI can be associated with any error codes reported from the sample. Both the CSI and the LSI can both be applied to the sample container.

Sample Plating. Once accessioned, the samples can be plated at step 214. At this point, the sample have been successfully integrated into the laboratory environment and are ready for analytics. At this point, the samples can be prepared for transfer to a sample microplate. The sample microplate can be labeled with a unique identifier, which can distinguish the sample microplate from other sample microplates. For example, the sample microplate can be a solid body with about 50 wells to about 400 wells, distributed across rows and columns, each well having a capacity of about 30 μL to about 300 μL. In other examples, different size microplates with a different number of wells at varying volumes can be used.

The samples to be used on the microplate may be racked and the rack may be assigned an identifier, such as to allow a technician to understand which samples correspond to which LSIs. The technician may unseal the sample, such as by a manual, automated, or semi-automated tool to efficiently open the sample container. The tooling may, for example, unscrew, cut, or drill each sample container, to make the sample within available for physical transfer to the sample microplate.

The samples can then be transferred to the microplate, such as by an automated robot that operates an end effector in accordance with one or more programs for effective transfer of the samples. This can be done, for example, with a combination of actuators, piezoelectric elements, pressure systems, and/or other components operating the end effector of the robot. The end effector can uptake portions of the samples in micropipettes and transfer those samples to the corresponding wells in the microplate. In some cases, disposable tips can be used. In some cases, portions of the samples can be transferred. In some cases, reagents can be added to the samples. In some cases, controls can be included in the microplate. The sample microplate, once completed, can be transferred for further processing in the laboratory.

Sample Storage. After plating, the samples can be stored at step 216. In some cases, accessioned samples, plated samples, or other samples, are stored for later use. In this case, they can be stored at room temperature, or can be cryogenically frozen and arranged on racks for later retrieval. Samples can be preserved for periods of days or years to allow later rapid re-testing.

Extraction of Genetic Material. When genetic analysis is desired, the genetic material of the samples can be extracted for sequencing at step 222. In some examples, a reagent can be applied to sample wells to lyse cells therein to expose genetic material.

Additionally, aspirating, and dispensing reagents can be used to selectively bind genetic material released from lysed cells. In some examples, this can include applying a bead to the well. In this case, the beads can, for example, be magnetic beads that selectively bind to the genetic material. This can help allow for isolation and purification of the genetic material at the bead, leaving contaminants in the solution. In an example, a magnetic bead can be magnetically drawn to a magnetic base at or under the sample microplate. In this case, after the genetic material has been drawn to the bead, a flushing step can be performed to wash away remaining fluid, helping to remove impurities.

In some examples, fluid can be added or removed from wells, such as to concentrate or elute the genetic material. Fluid can be transferred from the wells of the sample microplate to a genome stock microplate. In an example, a portion of fluid can be removed from each well for quality control purposes. This can, for example, be used to determine concentration of genetic material therein.

Library Preparation. After extraction of the genetic material, a library can be prepared using the contents of the genome stock microplate at step 224. For example, the bead for each well, including ionically bonded genetic material, can be transferred to a distinct well of a library preparation microplate. The library preparation microplate can include an identifier. The LSI associated with each well on the sample microplate can be mapped to a corresponding well on the library preparation microplate. The library preparation microplate may be transferred to a new portion of the laboratory to help prevent amplified genetic material from entering portions of the laboratory where genetic material has not been amplified, which could result in contamination.

A reagent can be applied to each well of the library preparation microplate. The reagent can ionically bond to the surface of the bead within the well more strongly than the genetic material. This helps release the genetic material from the surface of the bead of each well, enabling the genetic material to be chemically interacted with.

Library preparation can include normalization of a concentration of genetic material in each well of the sample microplate. Library preparation can further include fragmentation of the genetic material via an enzyme or via the application of physical forces. During this process, the entire genome (e.g., roughly three billion base pairs for a human genome), may be fragmented into pieces. In an example, the pieces can be about 300 to 400 base pairs in length. These pieces can be referred to as nucleic acid fragments. These nucleic acid fragments can undergo adaptor ligation and indexing. In an example, this can include Next Generation Sequencing (NGS) library preparation processes.

The genetic material can then be amplified, such as by Polymerase Chain Reaction (PCR) amplification. The resulting solution can be purified and eluted. During this library preparation, one or more reference samples of genetic material can be added to the wells of the library preparation microplate. The reference samples can serve as controls and aid in quality control.

Once the library preparation has been completed, thousands or millions of distinct fragments of the genetic material, each corresponding with a different portion of a genome of the subject, can be ligated to predefined adapters that bind with the genetic material. Each of the adaptor ligated fragments is referred to as a “library.”

In additional examples, probes applied to each well can include chemical identifiers (“barcodes”) that are distinct from each other. The use of a different chemical identifier for probes applied to each well of the well plate can enable sequencing to later be performed for multiple subjects on the same flow cell, without conflating sequencing results for those subjects.

In additional examples, the library preparation process can further include controlling a concentration of the genetic material in each well, and purification and/or elution of the resulting material. Similar to the processes performed after extraction of genetic material, concentration of genetic material after library preparation can be confirmed for each well via testing.

Enrichment of Genetic Material. After library preparation, enrichment processes can be performed in order to either directly amplify (e.g., via amplicon or multiplexed PCR) or capture (e.g., via hybrid capture) predefined libraries of genetic material, such as at step 226 in FIG. 2C. This can enhance the ease of sequencing desired portions of the genome.

Here, mitochondrial probes can be used during genetic sample enrichment, prior to amplification, to capture mitochondrial DNA (mtDNA). Pathogen genetic material is also captured, via the use of separate, pathogen-specific probes, such as in a viral assay. The mtDNA is amplified and sequenced along with the pathogen genetic material. The sequenced mtDNA is collected and called to produce a plurality of reads. The plurality of reads are aligned to a mitochondrial contig (a set of overlapping DNA segments that together represent a consensus region of DNA).

For example, during enrichment, customized biotinylated oligonucleotide probes can be applied to the libraries. The probes can selectively hybridize genetic material occupying desired portions of the genome for the genetic material, such as specific genes, or the entire exome. Magnetic beads can bind to biotin molecules in the probes to attach the hybridized material to the magnetic beads. Magnetic forces can capture the beads in place, enabling remaining fluid within each well to be removed or washed out, thereby removing impurities, and leaving only the genetic material that is desired. Thus, genetic material can be released from the beads in a similar manner to that discussed above for prior processes.

In an example, hybrid capture target enrichment can be performed. During this process, the probes can include tailored oligonucleotides that are chosen to bind to the genetic material. The range of probes can be tailored as a group to bind to specific alleles, specific genes, the exome, the entire genome, etc. That is, each probe can bind to a nucleic acid fragment at a specific location on the genome, and the range of probes can be selected to ensure that alleles, genes, the exome, or the entire genome of the subject being considered is acquired.

In these examples, utilizing probes in this manner can enhance efficiency of the sequencing process, by foregoing the need to sequence all of the roughly three billion base pairs found in the human genome. The enrichment process can further include controlling a concentration of the genetic material in each well, and purification and/or elution of the resulting material. Similar to the processes performed after extraction of genetic material, concentration of genetic material after enrichment can be confirmed for each well via testing.

Sequencing of Genetic Material. After enrichment, the genetic material can be sequenced at step 228. Sequencing can be performed according to any of a variety of techniques, including short-read and long-read techniques.

In an example, the sequencing can be performed as Sequencing by Synthesis (SBS) at genetic analyzer equipment. For example, sets of enriched libraries of genetic material bound to probes in earlier steps can be transferred to a flow cell, and annealed to oligonucleotide probes within the flow cell. At this stage, the contents of multiple wells can be applied to the same flow cell, because the libraries within those wells are tagged with the chemical identifiers referred to above.

In an example, the chemical identifiers can include nucleotide sequences that are detectable during the sequencing process to determine a corresponding LSI. Complementary sequences can then be created via enzymatic extension to create a double-stranded portion of genetic material. The double-stranded genetic material can then be denatured, and the library fragment can be washed away. Bridge amplification can then be performed to create copies of the remaining molecule in a localized cluster. For example, a cluster can comprise twenty to fifty copies of the same molecule, localized to a location the size smaller than a pinhead on the flow cell. Sequencing primers can be annealed to library adapters to prepare the flow cell for SBS. During SBS, the sequencing primer uses reverse terminator fluorescent oligonucleotides, one base per cycle, for several cycles in the forward direction. After the addition of each nucleotide, clusters can be excited by a light source, resulting in fluorescence which can be measured. The emission wavelength and signal intensity for each cluster determines a base call for that cluster. A chemical group blocking a 3′end of the fragment can then be removed, enabling a subsequent nucleotide to be read. This can help control nucleotide addition and detection. After each cycle, denaturing and annealing can be performed to extend the index primer. A complementary reverse strand can be created and extended via bridge amplification. The reverse strand can then be read in the reverse direction for a number of cycles, in a manner similar to reads in the forward direction.

Depending on whether a complete human genome, or another set of genomic data, is being tested, different reagents can be chosen. That is, different reagents can be utilized for library preparation for a pathogen (e.g., bacteria, virus) or an organelle (e.g., mitochondria) than for a human genome. Pathogens exhibiting Ribonucleic Acid (RNA) genomes can have their genetic material translated to DNA before sequencing, enrichment, and/or library preparation are performed.

In some examples, genetic material can be used for detection of a pathogen rather than for sequencing. Detecting a pathogen can involve the use of a real-time PCR system that performs PCR. The real-time PCR system can further add a reactive agent to individual wells of a library preparation microplate, that fluoresces when bound to genetic material for the pathogen. By analyzing fluorescence at known periods of time after PCR has initiated, presence of a pathogen is determined. Genetic testing for a pathogen can thereby forego sequencing in some examples.

Throughout the processes discussed above, the laboratory environment can be carefully controlled to ensure quality. For example, temperature within each segment of the laboratory can be carefully monitored and controlled, and ultraviolet lighting or other features capable of inactivating genetic material can be carefully positioned to ensure that contamination does not occur.

In general, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text based FASTQ format, that reports quality scores. The second format is then analyzed to perform alignment of sequence reads to a reference genome, such as a reference genome reported in a Browser Extensible Data (BED) file. The aligned sequence data may be reported as a Binary Alignment Map (BAM) file. The aligned sequence data may then be called, resulting in a Variant Call Format (VCF) file reporting called variants at each location of the genome that was sequenced, together with secondary metrics such as quality indicator metrics. The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Determination of Endogenous Control Presence and Contamination Status

After the samples have been received, processed, and sequenced in the wet laboratory environment 130 (see FIG. 1A and FIGS. 2A-2C), bioinformatic analysis can be done on the genetic data sequenced from the biological sample. An example method of bioinformatic analysis including determining an endogenous control and determining whether a sample is contaminated is outlined and discussed with reference to FIGS. 3A to 3E below. The method of FIG. 3A is an overarching method that includes each of the more detailed methods of FIGS. 3B to 3E.

FIG. 3A depicts method 302, which can include steps 312 to 316. First, at step 312, the bioinformatic laboratory 140 can receive genetic data sequenced from the biological sample from the wet lab 130. The genetic data can include data sequenced from the mtDNA, in addition to any data sequenced from the pathogen genetic data of interest. This is discussed in depth with reference to FIG. 3B below. Next, at step 314, the endogenous control can be satisfied, shown and discussed in detail with reference to FIG. 3C below. At step 316, whether or not the sample is contaminated is determined. This is discussed in detail with reference to FIGS. 3D and 3E below. The overarching method 302, shown here in FIG. 3A, incorporates both satisfying an endogenous control and determining whether the sample is contaminated.

As discussed above, the mtDNA can be collected with the use of mitochondrial probes during the sample enrichment phase, prior to amplification. FIG. 3B depicts method part 304, related to the collection of genetic material for sequencing. The part 304 includes wet lab steps, such as those described above with reference to FIGS. 2A to 2C. The part 304 of the method includes the physical collection, amplification, and enrichment of mtDNA for sequencing alongside the pathogen genetic material of interest.

Here, at step 322, the mtDNA is collected, such as with mitochondrial probes included in the genetic assay of the sample. In an example, a viral respiratory assay, targeted towards 29 viruses, can be used alongside mitochondrial probes in the biological sample. As needed, the probe depth can be adjusted to capture the mtDNA alongside the targeted pathogens. At step 324, the collected mtDNA is enriched, as discussed above. At step 326, the mtDNA is sequenced, using techniques such as described above. After this mtDNA is sequenced, alongside the pathogen genetic material, data is provided for bioinformatic analysis in the bioinformatic laboratory 140.

FIGS. 3C to 3E depicts portions of the method 302 that include steps done in the bioinformatic laboratory 140. Here, the genetic data sequenced from the biological sample, including genetic data sequenced from the capture mtDNA, has already been produced from the biological samples. The portions of the method discussed with reference to FIGS. 3C to 3E occur in the bioinformatic laboratory 140, not in the wet lab 130.

First, the endogenous control is satisfied, as shown in FIG. 3C. Here, in method 306, genetic data sequenced from the mtDNA is analyzed and used as an endogenous control (e.g., to confirm that this is a human sample containing human genetic material in the form of mtDNA). To confirm the endogenous control, sequenced genetic data is reviewed to confirm the presence of the mtDNA. If at least a threshold amount of the mitochondrial genome is present (e.g., at least 20% of the mitochondrial genome having at least ten reads), the endogenous control is positive. Shown in FIG. 3C, the method 306 can include several steps, including producing a plurality of mtDNA reads at step 332, aligning mtDNA reads to the mitochondrial contig at step 334, and checking the percent of the mitochondrial contig compared to a threshold at step 336.

First, a plurality of mtDNA reads can be produced (step 332). The sequenced genetic data, including the mtDNA data can be received and reviewed. A plurality of mtDNA reads can be produced based on genetic data sequenced from the mtDNA, and variants can be called. These reads can be used in an alignment step to verify whether the sample contains a reasonable amount of mtDNA, and therefore satisfies the endogenous control.

At the alignment step (step 334), the sequenced mtDNA reads can be aligned across the full mitochondrial contig (e.g., genome) to compare whether they match to a mitochondrial reference sequence. At this step, the various reads can be compared to the mitochondrial reference sequence (“chromosome M”) to confirm where or if those reads match. At this point, aligning the reads to chromosome M can help confirm whether the biological sample indeed contained mtDNA and came from a human patient. For example, if a large percentage of the mtDNA contig is represented, the biological sample likely came from a human patient.

With this in mind, the precent of the mitochondrial contig is compared to a threshold value (step 336). If a minimum amount of the mitochondrial genome is covered by a predetermined number of reads, then the presence of human genetic material in the sample is confirmed, and the endogenous control is satisfied. In an example, if a minimum of 20% of the mitochondrial genome is covered over at least ten reads produced from the genetic data sequenced from the mtDNA, the endogenous control can be satisfied.

Once the endogenous control is satisfied, the method can further include determining whether the sample has been contaminated. FIG. 3D and FIG. 3E depict these methods 308 and 310. Here, alternative allele fraction calculations can be used to aid in determining whether the biological sample was contaminated.

First, shown in FIG. 3D, variants across the mtDNA can be called. At step 342, an alternative allele fraction can be calculated for each of multiple specified loci on the mitochondrial genome. For a specific mutation, an alternative allele fraction is the number of reads from the sample that support that mutation at that locus, divided by the total number of reads in the sample at that locus. An AAF can be calculated for each of the loci of interest on the mitochondrial contig.

Next, at step 344, it can be determined whether the alternative allele fraction is above a minimum threshold, and therefore may be called as a variant. This can allow for calling of variants at step 346. Mitochondrial DNA is haploid, therefore all or most of the reads at a specific locus should support the reference base at that position, or alternatively, all or most of the reads should support a different variant. Any variants present, indicated by the AAF calculated at the loci of interest, can be called.

Shown in FIG. 3E, whether or not a sample is contaminated can be determined. First, at step 352, the calculated alternative allele fractions for called variants can be used to produce one or more composite alternative allele fraction-based calculations which represent the sample as a whole. Such a calculation could include a median, average, or mean alternative allele fraction. For example, a median AAF can be calculated by taking the median of the AAFs across all loci in the sample which have an AAF above a threshold minimum amount. For example, a median AAF can be calculated using all loci in the sample that have an AAF above about 0.15 or about 0.20.

At step 354, the composite alternative allele fraction-based calculations can be compared to a composite AAF threshold in order to determine the likelihood of contamination. Taking the alternative allele fraction calculation for a single given mtDNA variant, a value of 0 or 1 would be expected for mitochondrial DNA in an un-contaminated sample. That is, for a specific biological sample from a single patient, that patient only has one, haploid set of mtDNA. For this reason, a particular locus of the mtDNA genome, the patient's mtDNA set would only be representative of one person; the variant would likely have a value of either 0 or 1, meaning the variant is either present or not. Similarly, when the composite AAF is calculated across all variant loci that have an AAF above a certain threshold, the composite AAF is expected to be close to or equal to 1 if only one human genetic source is present. At step 356, a determination is made whether contamination is present based on the calculated composite AAF. For example, if the calculation is below a predetermined threshold, e.g., 0.95 or 0.98, the sample is likely contaminated. That is, the sample could contain mtDNA from more than one patient, which would account for the variants of mtDNA.

To summarize, mtDNA can aid in confirming that a human specimen was present in the sequenced sample in the absence of a pathogen positive and also help determine whether it is likely that the biological sample contains genetic material from more than one individual. Thus, the use of mitochondrial probes and genetic material analysis can allow for contemporaneous confirmation of an endogenous control and checking for contamination, simplifying the process overall.

### Computer Example

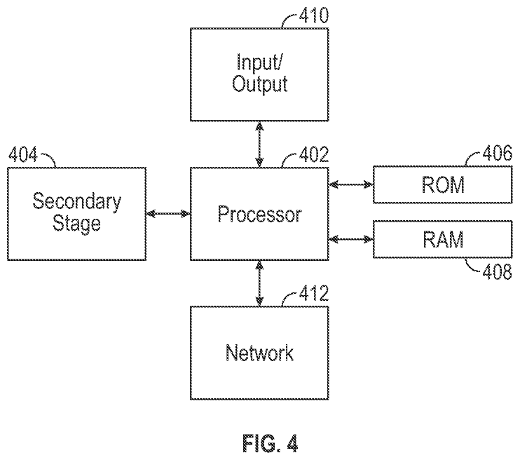

FIG. 4 is a block diagram of a typical, general-purpose computer 400 that may be programmed into a special purpose computer suitable for implementing one or more examples of the manifest record generating program disclosed herein. The manifest record generating program described above may be implemented on any general-purpose processing component, such as a computer with sufficient processing power, memory resources, and communications throughput capability to handle the necessary workload placed upon it. The computer 400 includes a processor 402 (which may be referred to as a central processor unit or CPU) that is in communication with memory devices including secondary storage 404, read only memory (ROM) 406, random access memory (RAM) 408, input/output (I/O) devices 410, and network connectivity devices 412. The processor 402 may be implemented as one or more CPU chips or may be part of one or more application specific integrated circuits (ASICs).

The secondary storage 404 is typically comprised of one or more disk drives or tape drives and is used for non-volatile storage of data and as an over-flow data storage device if RAM 408 is not large enough to hold all working data. Secondary storage 404 may be used to store programs that are loaded into RAM 408 when such programs are selected for execution. The ROM 406 is used to store instructions and perhaps data that are read during program execution. ROM 406 is a non-volatile memory device that typically has a small memory capacity relative to the larger memory capacity of secondary storage 404. The RAM 408 is used to store volatile data and perhaps to store instructions. Access to both ROM 406 and RAM 408 is typically faster than to secondary storage 404.

The devices described herein may be configured to include computer-readable non-transitory media storing computer readable instructions and one or more processors coupled to the memory, and when executing the computer readable instructions configure the computer 400 to perform method steps and operations described above with reference to FIG. 3 to FIG. 4. The computer-readable non-transitory media includes all types of computer readable media, including magnetic storage media, optical storage media, flash media and solid-state storage media.

It should be further understood that software including one or more computer-executable instructions that facilitate processing and operations as described above with reference to any one or all of steps of the disclosure may be installed in and sold with one or more servers and/or one or more routers and/or one or more devices within consumer and/or producer domains consistent with the disclosure. Alternatively, the software may be obtained and loaded into one or more servers and/or one or more routers and/or one or more devices within consumer and/or producer domains consistent with the disclosure, including obtaining the software through physical medium or distribution system, including, for example, from a server owned by the software creator or from a server not owned but used by the software creator. The software may be stored on a server for distribution over the Internet, for example.

Also, it will be understood by one skilled in the art that this disclosure is not limited in its application to the details of construction and the arrangement of components set forth in the description or illustrated in the drawings. The examples herein are capable of other examples, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated examples may be implemented, at least in part, in digital electronic circuitry, analog electronic circuitry, or in computer hardware, firmware, software, or in combinations of them. These components may be implemented, for example, as a computing program product such as a computing program, program code or computer instructions tangibly embodied in an information carrier, or in a machine-readable storage device, for execution by, or to control the operation of, data processing apparatus such as a programmable processor, a computer, or multiple computers.

A computing program may be written in any form of programming language, including compiled or interpreted languages, and it may be deployed in any form, including as a stand-alone program or as a module, component, subroutine, or other unit suitable for use in a computing environment. A computing program may be deployed to be executed on one computer or on multiple computers at one site or distributed across multiple sites and interconnected by a communication network. Also, functional programs, codes, and code segments for accomplishing the techniques described herein may be easily construed as within the scope of the present disclosure by programmers skilled in the art. Method steps associated with the illustrative examples may be performed by one or more programmable processors executing a computing program, code or instructions to perform functions (e.g., by operating on input data and/or generating an output). Method steps may also be performed by, and apparatus may be implemented as, special purpose logic circuitry, e.g., an FPGA (field programmable gate array) or an ASIC (application-specific integrated circuit), for example.

The various illustrative logical blocks, modules, and circuits described in connection with the examples disclosed herein may be implemented or performed with a general-purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computing program include, by way of example, both general and special purpose microprocessors, and any one or more processors of any kind of digital computer. Generally, a processor will receive instructions and data from a read-only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computing program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory may be supplemented by or incorporated in special purpose logic circuitry.

Those of skill in the art understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill in the art further appreciate that the various illustrative logical blocks, modules, circuits, and algorithm steps described in connection with the examples disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the disclosure. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor may read information from, and write information to, the storage medium. In the alternative, the storage medium may be integral to the processor. In other words, the processor and the storage medium may reside in an integrated circuit or be implemented as discrete components.

As used herein, “machine-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” should be taken to include a single medium or multiple media (e.g., a centralized or distributed database, or associated caches and servers) able to store processor instructions. The term “machine-readable medium” shall also be taken to include any medium, or combination of multiple media, that is capable of storing instructions for execution by one or more processors, such that the instructions, when executed by one or more processors cause the one or more processors to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” as used herein excludes signals per se.

### Examples

Various examples of the present disclosure can be better understood by reference to the following Examples which are offered by way of illustration. The present disclosure is not limited to the Examples given herein.

The following sequencing and analysis process were applied to all samples described in the Examples below. The biological samples were collected and sequenced as outlined above with reference to FIGS. 2A to 2C. The mitochondrial probes (102040: Twist Mitochondrial Panel, 96 Reactions, Kit) were added to the Samples after library amplification of the genetic material, but before enrichment amplification. The mitochondrial probes were applied together with the viral panel probes used for sequencing respiratory pathogens, at a concentration appropriate to capture, sequence, and call variants across the entire mitochondrial contig.

The genetic sequence data produced from the collected mtDNA was analyzed for an endogenous control. Alignments were produced and coverage of the mitochondrial contig was computed with an alignment step. Minimum coverage of the mitochondrial contig was identified to confirm presence of the endogenous control in the Samples. Subsequently, the Samples were analyzed for contamination.

### Example 1. AAFs of Individual Mitochondrial Variant Sites in Uncontaminated Samples and in Silico Sample Mixtures

Human mtDNA is inherited maternally and is as a rule haploid. When mtDNA is sequenced from an individual, the general expectation is that the alternative allele fractions of any variants called will be close to 1, with some possible noise due to sequencing error or occasional mutations.

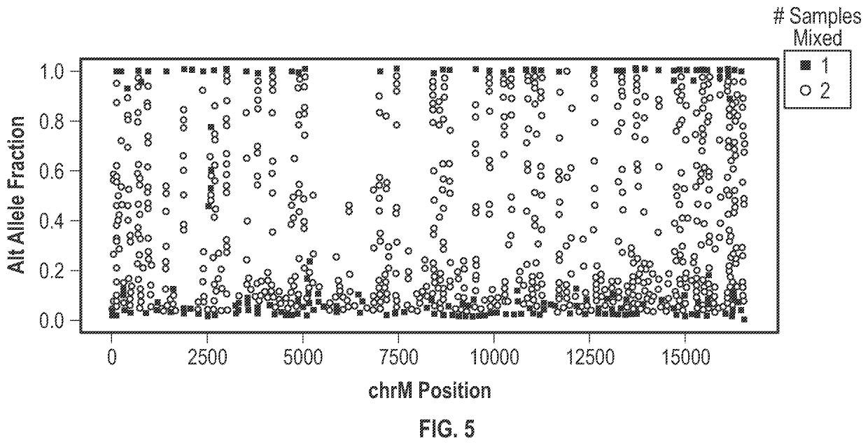

By contrast, if a sequenced sample contains many variants with mixed allele fractions, the most likely explanation is that the sample contains mtDNA from more than one individual, in other words, is contaminated. This is illustrated in FIG. 5, which shows the alternative allele fraction (y-axis) against the mitochondrial chromosome position (x-axis).

Shown in FIG. 5 are alternative allele fractions of mitochondrial variants in two samples that each come from only one human patient (“1”, squares) compared to an in silico mixture created by combining the genetic data from those same two samples after sequencing but before bioinformatic analysis (“2”, circles). Most of the AAFs in the individual samples (squares) have values close to either 1 or 0, whereas the AAFs in the mixed sample (circles) often take on intermediate values between 0 and 1, illustrating the expected contamination pattern.

### Example 2. Median AAF in Individual Samples and in Silico Sample Mixtures

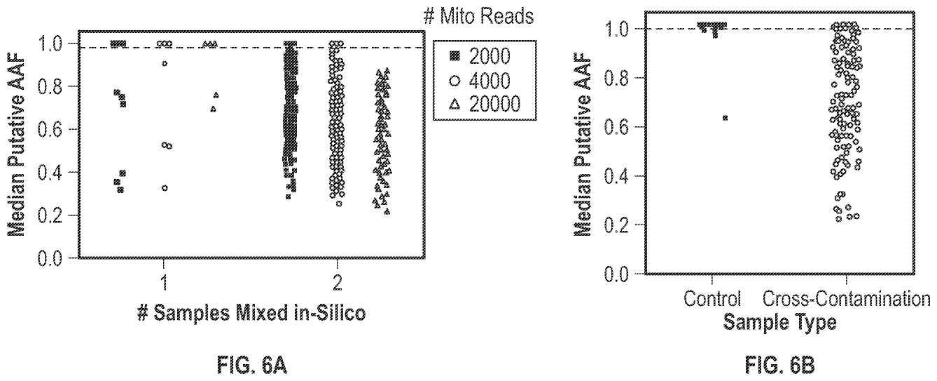

In this example, 283 samples from individual patients were sequenced. Then, in silico mixtures were created by bioinformatically combining the mitochondrial genetic data from pairs of samples (to simulate contamination). Different mixture ratios were used (50:50, 75:25, 80:20) to simulate different levels of contamination. The alignments from the individual samples and the mixtures were then randomly down sampled to 2000, 4000, and 20,000 reads in order to simulate the effect of lower or higher yields on the mitochondrial AAF values. In total, 2,010 mixtures were created. After this processing, the steps described in FIG. 3D and FIG. 3E were performed on each individual sample and each mixture. FIG. 6A shows the median (composite) AAF value of these samples, with the marker shape denoting the number of reads in the sample (square: 2000, circle: 4000, or triangle: 20,000), and the X-axis label describing whether the sample is individual (“1”) or mixed to simulate contamination (“2”).

The data for the individual samples (“1”) show median AAF values that hover mostly at or near 1, as expected, indicating that contamination is unlikely. By contrast, the median AAF values from the in silico mixtures (“2”) vary widely, covering the range between 0 and 1. A median AAF threshold value of 0.98 is sufficient to distinguish the individual samples from the mixtures in most cases, demonstrating the utility of this metric for detecting contamination. Although the separation is most evident when the number of reads is high (20,000), the general difference in median AAF between individual and mixed samples can still be observed at lower read counts.

### Example 3: Median AAF in Individual and Cross-Contaminated Samples

An additional AAF experiment was performed by intentionally contaminating 192 samples in the laboratory and comparing them to their uncontaminated counterparts. FIG. 6B shows the median AAF values in the uncontaminated samples (“Control) alongside those of the contaminated samples (“Cross-Contamination”). Together, FIG. 6A and FIG. 6B show that a 0.98 threshold for the median AAF effectively distinguishes most in silico and experimental mixtures from uncontaminated samples. Thus, median alternative allele fraction from mtDNA is a reasonable metric to use for detecting contamination in this assay.

The terms and expressions that have been employed are used as terms of description and not of limitation, and there is no intention in the use of such terms and expressions of excluding any equivalents of the features shown and described or portions thereof, but it is recognized that various modifications are possible within the scope of the examples of the present disclosure. Thus, it should be understood that although the present disclosure has been specifically disclosed by specific examples and optional features, modification and variation of the concepts herein disclosed may be resorted to by those of ordinary skill in the art, and that such modifications and variations are considered to be within the scope of examples of the present disclosure.

### VARIOUS NOTES & EXAMPLES

Example 1 is a method of performing quality control for a biological sample, the method comprising: receiving genetic data sequenced from the biological sample, wherein the genetic data has been analyzed for both pathogen genetic material and mitochondrial genetic material; determining whether an endogenous control is apparent in the genetic data based on whether at least a threshold amount of the genetic data includes sequences for mitochondrial genetic material; and if the endogenous control is apparent in the genetic data, determining whether the biological sample is contaminated based on calculating a composite alternative allele fraction of the sequences for the mitochondrial genetic material.

In Example 2, the subject matter of Example 1 optionally includes wherein calculating the composite alternative allele fraction based on the mitochondrial genetic material comprises calculating a median, average, or mean alternative allele fraction, based on calculated alternative allele fractions at loci for called mitochondrial genetic variants.

In Example 3, the subject matter of any one or more of Examples 1-2 optionally include wherein calculating the composite alternative allele fraction based on the mitochondrial genetic material comprises determining whether the composite alternative allele fraction is over a predetermined threshold.

In Example 4, the subject matter of Example 3 optionally includes wherein the predetermined threshold is 0.95.

In Example 5, the subject matter of any one or more of Examples 3-4 optionally include determining that the biological sample is contaminated if the composite alternative allele fraction is below the predetermined threshold.

In Example 6, the subject matter of any one or more of Examples 1-5 optionally include wherein determining whether the endogenous control is apparent comprises reviewing the genetic data to confirm that human genetic material is in the biological sample.

In Example 7, the subject matter of any one or more of Examples 1-6 optionally include wherein the threshold amount comprises at least twenty percent of a mitochondrial genome covered by at least ten reads.

In Example 8, the subject matter of any one or more of Examples 1-7 optionally include sequencing the biological sample.

In Example 9, the subject matter of Example 8 optionally includes wherein sequencing the biological sample comprises sequencing the mitochondrial genetic material from the biological sample contemporaneously with the pathogen genetic material from the biological sample.

In Example 10, the subject matter of any one or more of Examples 8-9 optionally include using one or more mitochondrial probes to collect the mitochondrial genetic material from the biological sample and enable amplification of the mitochondrial genetic material.

In Example 11, the subject matter of Example 10 optionally includes wherein the using of the one or more mitochondrial probes is performed during enrichment of genetic material of the biological sample and prior to amplification of genetic material of the biological sample.

In Example 12, the subject matter of any one or more of Examples 8-11 optionally include capturing the mitochondrial genetic material from the biological sample and sequencing the mitochondrial genetic material.

In Example 13, the subject matter of Example 12 optionally includes producing a plurality of reads based on the mitochondrial genetic material, wherein the plurality of reads extend across a mitochondrial genome.

In Example 14, the subject matter of Example 13 optionally includes aligning the plurality of reads to a mitochondrial contig to determine whether the endogenous control is apparent.

In Example 15, the subject matter of Example 14 optionally includes wherein determining whether an endogenous control is apparent comprises determining whether at least 20% of the mitochondrial contig is present in the plurality of reads.

In Example 16, the subject matter of any one or more of Examples 1-15 optionally include wherein, if less than the threshold amount of the genetic data includes sequences for the mitochondrial genetic material is present in the sample, the endogenous control is not apparent.

In Example 17, the subject matter of any one or more of Examples 1-16 optionally include calling mitochondrial genetic variants of the sequences for the mitochondrial genetic material, across a mitochondrial genome for the mitochondrial genetic material, wherein calling mitochondrial genetic variants comprises: calculating an alternative allele fraction at each of multiple loci across the mitochondrial genome; for each of the loci, determining whether the alternative allele fraction is above a minimum threshold; and for each of the loci, if the alternative allele fraction is above the minimum threshold, calling a mitochondrial genetic variant at the loci.

Example 18 is a system for quality control of a biological sample, the system comprising a processor and a memory that holds instructions which when executed cause the processor to: access genetic data sequenced from the biological sample, wherein the genetic data has been analyzed for both pathogen genetic material and mitochondrial genetic material; determine whether an endogenous control is apparent in the genetic data based on whether at least a threshold amount of the genetic data includes sequences for the mitochondrial genetic material; calculate a composite alternative allele fraction of the sequences for the mitochondrial genetic material; and determine whether the biological sample is contaminated based on the calculated composite alternative allele fraction.

In Example 19, the subject matter of Example 18 optionally includes wherein causing the processor to calculate the composite alternative allele fraction based on the mitochondrial genetic material comprises calculating a median, average, or mean alternative allele fraction, based calculated alternative allele fractions at loci for called mitochondrial genetic variants.

In Example 20, the subject matter of any one or more of Examples 18-19 optionally include wherein causing the processor to calculate the composite alternative allele fraction based on the mitochondrial genetic material comprises determining whether the composite alternative allele fraction is over a predetermined threshold.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method of performing quality control for a biological sample, the method comprising:
receiving genetic data sequenced from the biological sample, wherein the genetic data has been analyzed for both pathogen genetic material and mitochondrial genetic material;
determining whether an endogenous control is apparent in the genetic data based on whether at least a threshold amount of the genetic data includes sequences for mitochondrial genetic material; and
if the endogenous control is apparent in the genetic data, determining whether the biological sample is contaminated based on calculating a composite alternative allele fraction of the sequences for the mitochondrial genetic material.

2. The method of claim 1, wherein calculating the composite alternative allele fraction based on the mitochondrial genetic material comprises calculating a median, average, or mean alternative allele fraction, based on calculated alternative allele fractions at loci for called mitochondrial genetic variants.

3. The method of claim 1, wherein calculating the composite alternative allele fraction based on the mitochondrial genetic material comprises determining whether the composite alternative allele fraction is over a predetermined threshold.

4. The method of claim 3, wherein the predetermined threshold is 0.98.

5. The method of claim 3, further comprising determining that the biological sample is contaminated if the composite alternative allele fraction is below the predetermined threshold.

6. The method of claim 1, wherein determining whether the endogenous control is apparent comprises reviewing the genetic data to confirm that human genetic material is in the biological sample.

7. The method of claim 1, wherein the threshold amount comprises at least twenty percent of a mitochondrial genome covered by at least ten reads.

8. The method of claim 1, further comprising sequencing the biological sample.

9. The method of claim 8, wherein sequencing the biological sample comprises sequencing any mitochondrial genetic material from the biological sample contemporaneously with pathogen genetic material from the biological sample.

10. The method of claim 8, further comprising using one or more mitochondrial probes to collect the mitochondrial genetic material from the biological sample and enable amplification of the mitochondrial genetic material.

11. The method of claim 10, wherein the using of the one or more mitochondrial probes is performed during enrichment of genetic material of the biological sample and prior to amplification of genetic material of the biological sample.

12. The method of claim 8, further comprising capturing the mitochondrial genetic material from the biological sample and sequencing the mitochondrial genetic material.

13. The method of claim 12, further comprising producing a plurality of reads based on the mitochondrial genetic material, wherein the plurality of reads extend across a mitochondrial genome.

14. The method of claim 13, further comprising aligning the plurality of reads to a mitochondrial contig to determine whether the endogenous control is apparent.

15. The method of claim 14, wherein determining whether an endogenous control is apparent comprises determining whether at least 20% of the mitochondrial contig is present in the plurality of reads.

16. The method of claim 1, wherein, if less than the threshold amount of the genetic data includes sequences for the mitochondrial genetic material is present in the sample, the endogenous control is not apparent.

17. The method of claim 1, further comprising calling mitochondrial genetic variants of the sequences for the mitochondrial genetic material, across a mitochondrial genome for the mitochondrial genetic material, wherein calling mitochondrial genetic variants comprises:
calculating an alternative allele fraction at each of multiple loci across the mitochondrial genome;
for each of the loci, determining whether the alternative allele fraction is above a minimum threshold; and
for each of the loci, if the alternative allele fraction is above the minimum threshold, calling a mitochondrial genetic variant at the loci.

18. A system for quality control of a biological sample, the system comprising a processor and a memory that holds instructions which when executed cause the processor to:
access genetic data sequenced from the biological sample, wherein the genetic data has been analyzed for both pathogen genetic material and mitochondrial genetic material;
determine whether an endogenous control is apparent in the genetic data based on whether at least a threshold amount of the genetic data includes sequences for the mitochondrial genetic material;
calculate a composite alternative allele fraction of the sequences for the mitochondrial genetic material; and
determine whether the biological sample is contaminated based on the calculated composite alternative allele fraction.

19. The system of claim 18, wherein causing the processor to calculate the composite alternative allele fraction based on the mitochondrial genetic material comprises calculating a median, average, or mean alternative allele fraction, based calculated alternative allele fractions at loci for called mitochondrial genetic variants.

20. The system of claim 18, wherein causing the processor to calculate the composite alternative allele fraction based on the mitochondrial genetic material comprises determining whether the composite alternative allele fraction is over a predetermined threshold.
